(19) United States
(12) Patent Application Publication
(10) Pub. No.: US 2018/0038842 A1
(43) Pub. Date: Feb. 8, 2018

(54) MICROFLUIDIC DEVICE AND APPARATUS COMPRISING SUCH A DEVICE

(71) Applicant: CENTRE NATIONAL DE LA RECHERCHE SCIENTIFIQUE - CNRS, Paris (FR)

(72) Inventors: Jacques Gierak, Le Plessis Pate (FR); Loïc Auvray, Montrouge (FR); Julien Chaste, Villejuif (FR)

(73) Assignee: CENTRE NATIONAL DE LA RECHERCHE SCIENTIFIQUE - CNRS, Paris (FR)

(21) Appl. No.: 15/552,214

(22) PCT Filed: Feb. 17, 2016
(86) PCT No.: PCT/FR2016/050361

§ 371 Date: Aug. 18, 2017

(30) Foreign Application Priority Data
Feb. 19, 2015 (FR) 15 51379

(51) Int. Cl. G01N 33/487; B01L 3/00

(52) U.S. Cl. CPC G01N 33/48721; B01L 3/502707; B01L 3/502715; B01L 3/50273

(57) ABSTRACT

The microfluidic device comprises a body and a covering sheet. The body comprises a base section that includes an exterior surface and a channel bottom which extends in a main longitudinal direction and which is formed in the base section in the exterior surface. The channel bottom is formed by a focused ion beam. The covering sheet is joined to the base section and covers the channel bottom.

The present invention relates to microfluidic devices and to apparatus comprising such devices.

An example of such an apparatus is an electrophoresis apparatus.

The principle of electrophoresis is based on the migration of species carrying a net electrical charge which move under the action of an electric field generated by applying a difference in potential applied to each side of a membrane pierced with a passage of nanometric transverse dimensions.

Such methods have been implemented using biological membranes. However, because of problems inherent to the use of such biological membranes, there are increasing efforts to use, where possible, a membrane provided with an artificial passage (or “synthetic” membrane).

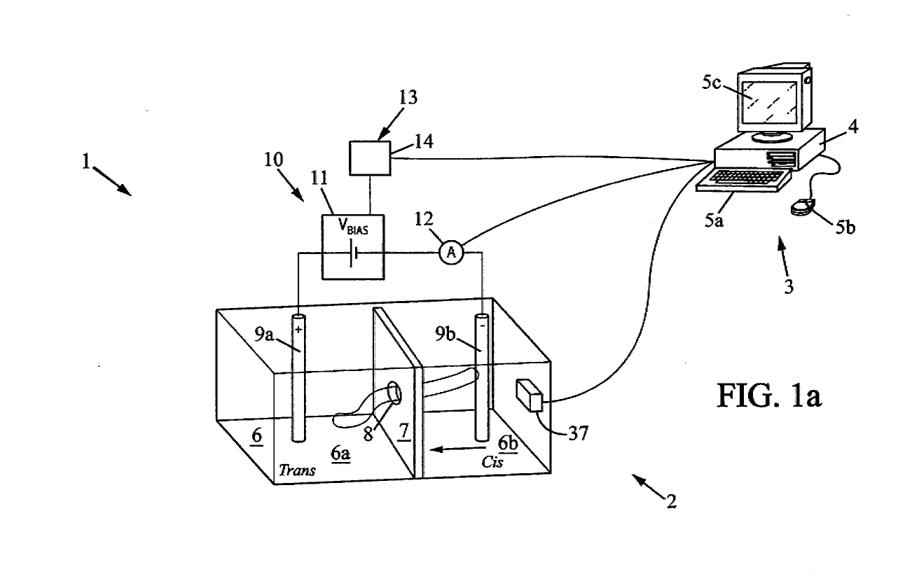

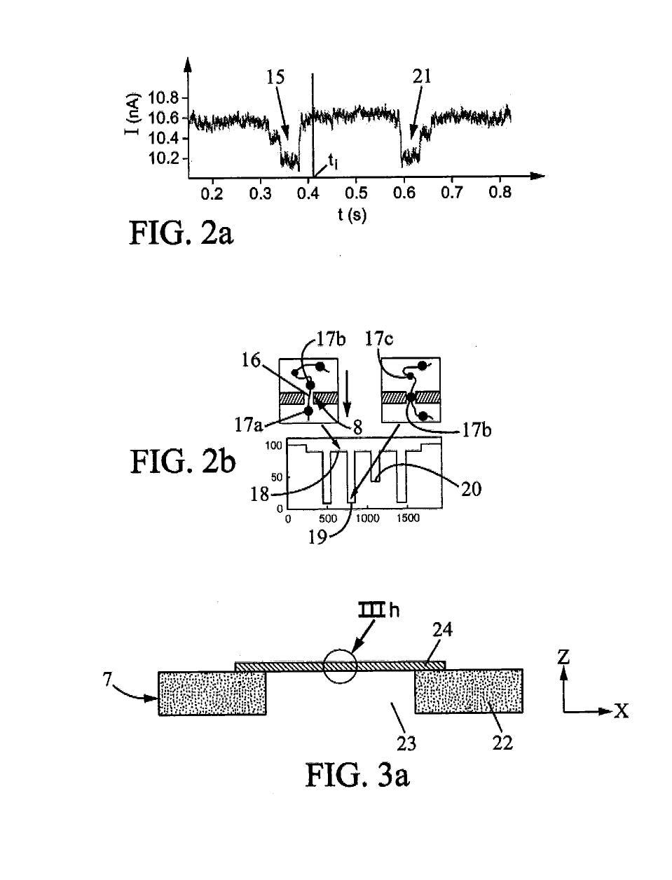

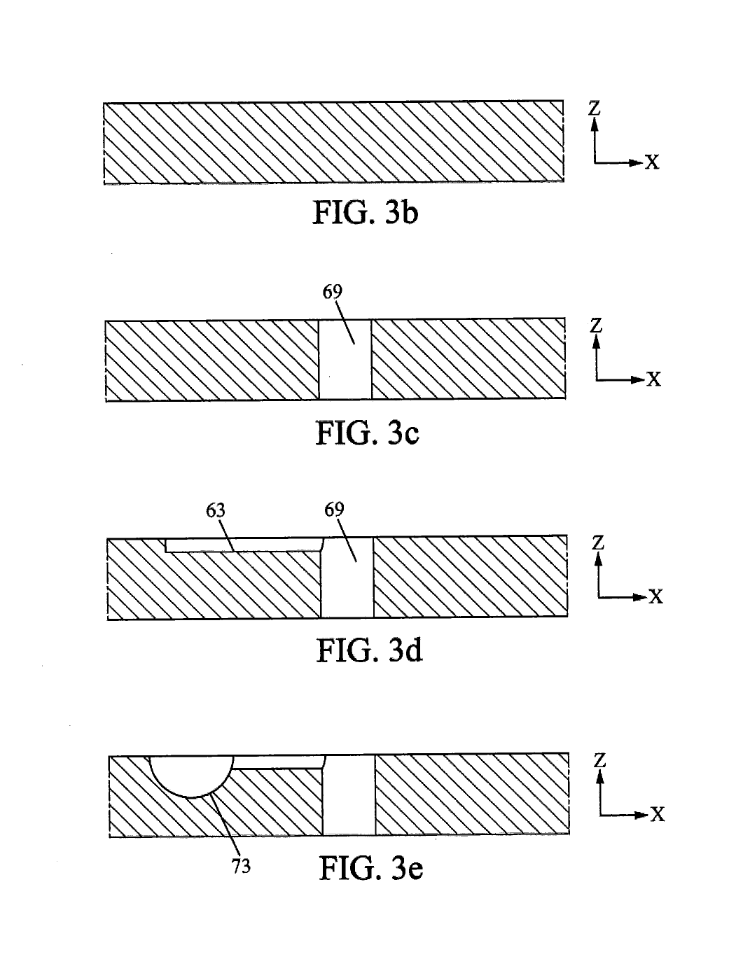

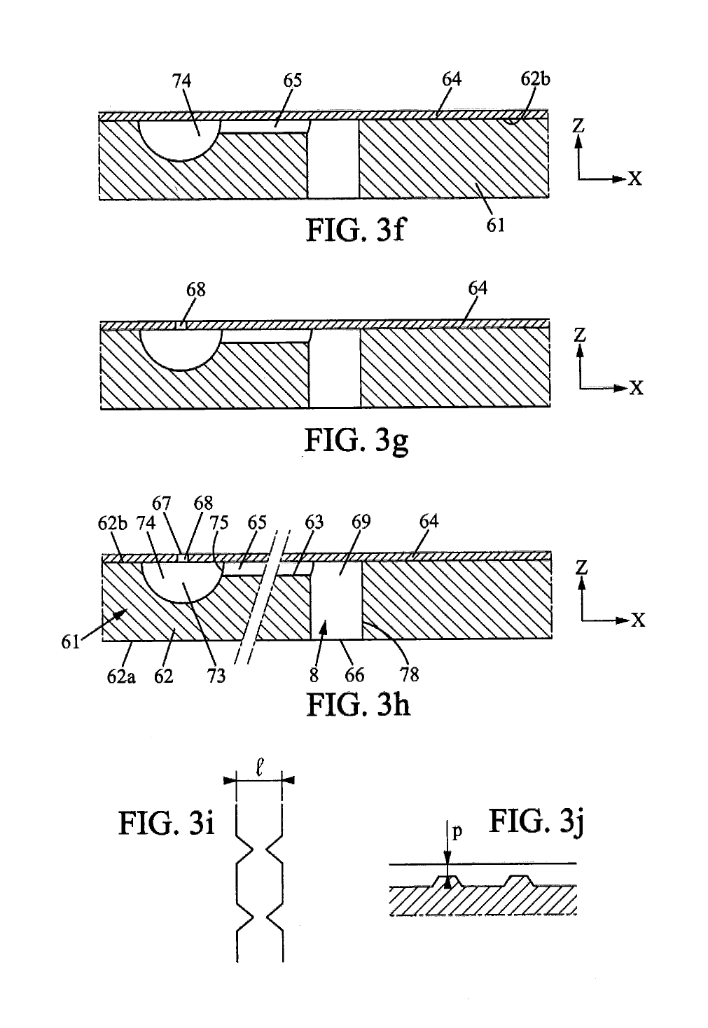

The document by B. Schiedt et al. entitled “Direct FIB fabrication and integration of ‘single nanopore devices’ for the manipulation of macromolecules”, Microelectron. Eng. (2010), doi: 10.1016/j.mee.2009.12.073 describes an example of such a device. An event, such as the migration of a macromolecule of solution through a pore, is detected by a decrease in the current detected by the reading system (FIGS. 3b and 3c of that document—see FIG. 2a of the present application).

At present, the only known information to be obtained using such a setup is binary information: the migration or non-migration of the macromolecule through the nanopore.

We seek to improve the detection of events.

To this end, according to the invention, a microfluidic device is provided comprising a body and a covering sheet,

the body comprising a base section having an outer surface, a channel bottom extending in a main longitudinal direction being formed in the base section in the outer surface, the channel bottom being formed by focused ion beam,

the covering sheet being joined to the base section while at least partially covering the channel bottom, thereby forming a channel.

With these features, detectability of the macromolecule in the passage is increased, which can be useful for many types of applications.

In some embodiments of the invention, one or more of the following arrangements may possibly be used:

the covering sheet comprises an electrically conductive layer and/or an electrically insulating layer, for example a superimposed electrically conductive layer and electrically insulating layer, in particular wherein a layer of the sheet comprises, in particular consists of, graphene, boron nitride (h-BN), or molybdenum disulfide (MoS₂) ;

the body comprises, in particular consists of, silicon or an oxide, carbide, or nitride of silicon;

the microfluidic device further comprises at least one electrode at least partially arranged in the vicinity of the channel;

the microfluidic device comprises an inlet end and an outlet end, both in fluid communication with the channel;

the inlet end and/or outlet end is part of a pore traversing the body and opening into the channel and extending in the thickness direction;

the channel opens into an inlet compartment and/or outlet compartment formed in the body;

the covering sheet covers the inlet compartment and/or outlet compartment;

the depth and/or width of the channel varies along the main longitudinal direction;

the body is a thin body less than 10 microns in thickness;

a well bottom is formed in the base section at the outer surface, the well bottom being formed by focused ion beam,

the covering sheet being joined to the base section while at least partially covering the well bottom, thereby forming a well in fluid communication with the channel.

According to another aspect, the invention relates to an apparatus comprising:

a reservoir adapted to receive an electrically conductive solution,

such a microfluidic device immersed in the reservoir and separating the reservoir into first and second compartments, the channel being in fluid communication with the first and second compartments to permit fluid communication between the first and second compartments,

a transport system adapted to generate movement of the solution in the reservoir when the reservoir contains the solution,

a system for characterizing the species contained in the reservoir.

In some embodiments of the invention, one or more of the following arrangements may possibly be used:

the transport system comprises an electrical system adapted to apply an electric field in the reservoir when the reservoir contains the solution,

the characterization system comprises a system for reading the electric field in the reservoir;

the apparatus further comprises a modulation system for modulating an electric field present in the channel;

the electrical system comprises first and second electrodes respectively arranged in the first and second compartments, between which the electric field is applied, the modulation system comprising said first and second electrodes which are reversible, and an inversion system adapted for reversing the polarity of an electric field applied between the two electrodes;

the modulation system comprises a set of local electrodes comprising at least said electrode, and a generator adapted to apply a local electric field at the channel via said set of at least one local electrode;

the apparatus further comprises an optical reading system adapted to take an image of the channel;

the apparatus comprises at least one of the following arrangements:

the microfluidic device (7) comprises a single passage,

the solution has a high concentration of solute and a low concentration of particles (27), the particles being potentially identical or possibly even a single particle, the transverse dimension of the particles possibly being between 0.5 and 0.9 times the transverse dimension of the channel.

Other features and advantages of the invention will be apparent from the following description of seven of its embodiments, given by way of non-limiting example, with reference to the accompanying drawings.

In the drawings:

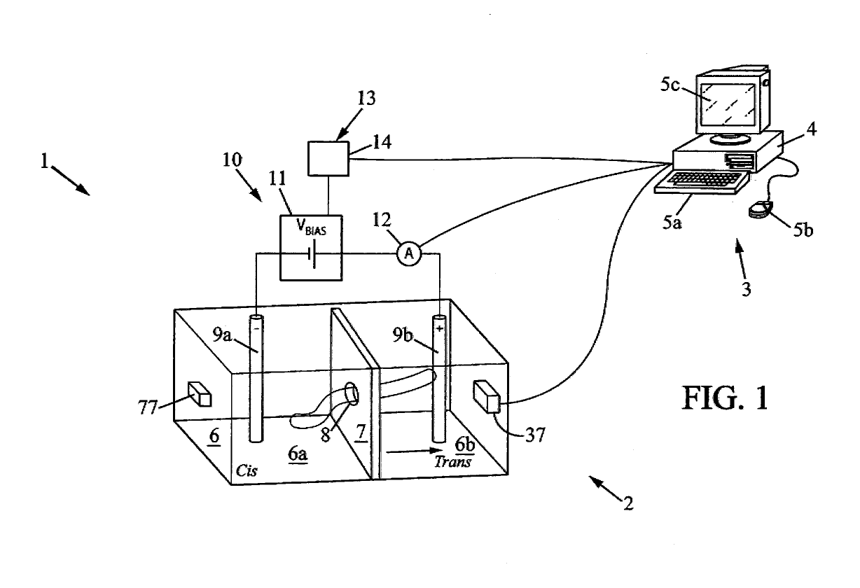

FIG. 1 is a schematic perspective view of an apparatus according to a first embodiment, used with a first polarity;

FIG. 1a is a view corresponding to FIG. 1 for the same apparatus used with a second polarity,

FIG. 2a is a graph showing the intensity of the ion current flowing between the two electrodes, and measured with the apparatus of FIG. 1 over time,

FIG. 2b is an explanatory diagram showing a simplified example of such a graph, schematically representing the migration of a macromolecule through the passage at two moments separated by time and thus blocking the passage in two different ways,

FIG. 3a is a macroscopic schematic sectional view of a membrane portion for the apparatus according to the first embodiment,

FIGS. 3b to 3g are microscopic schematic views showing different successive steps in the fabrication of the sheet equipping the membrane,

FIG. 3h is an enlargement (of portion IIIh) of FIG. 3a,

FIG. 3i is a schematic top view of an alternative embodiment,

FIG. 3j is a schematic sectional view of an alternative embodiment,

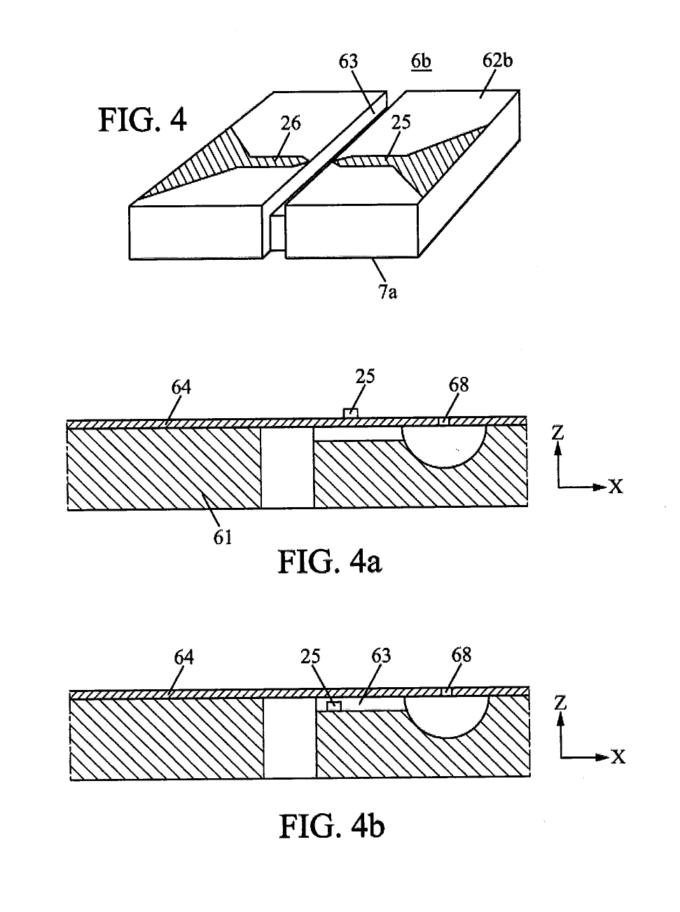

FIG. 4 is a schematic view of a membrane portion according to a second embodiment,

FIGS. 4a and 4b are partial sectional views in the same section plane as FIG. 3h, for two variants with integrated electrode,

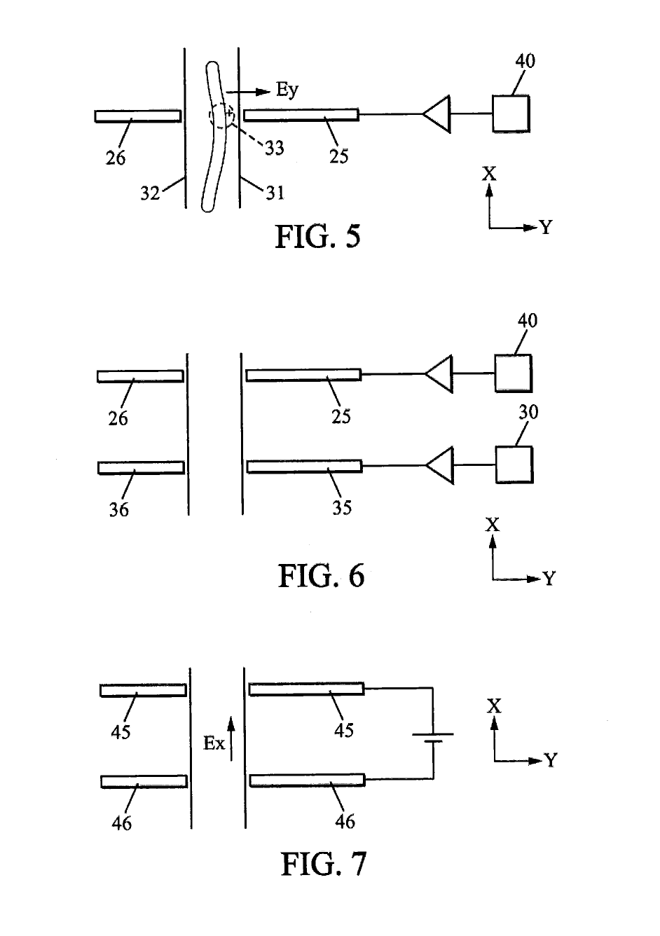

FIGS. 5, 6, and 7 are views corresponding to FIG. 4, respectively for the fourth, fifth, and sixth embodiments of the invention,

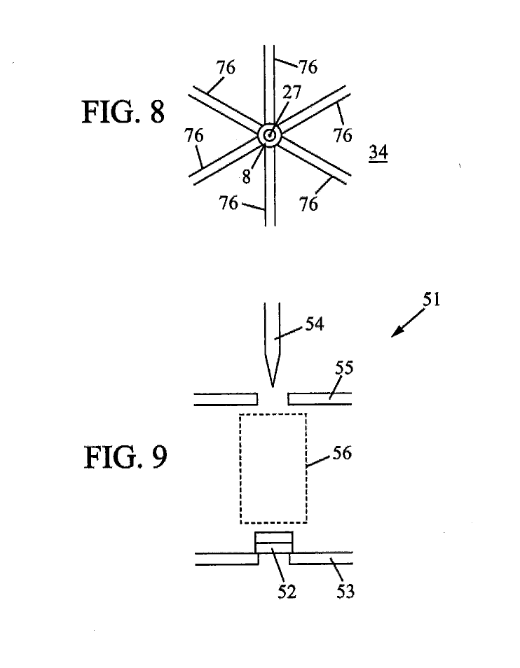

FIG. 8 is a top view of the membrane according to a seventh embodiment,

FIG. 9 is a schematic view of an apparatus for manufacturing for such a membrane,

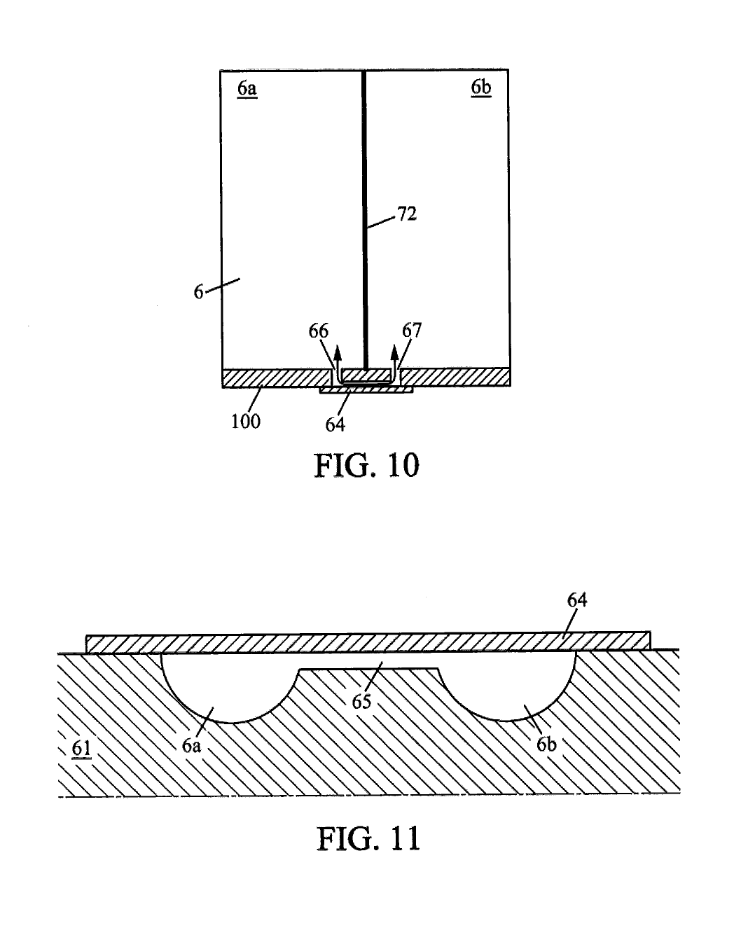

FIG. 10 shows an alternative embodiment of FIG. 1,

FIG. 11 is a sectional view of an embodiment of the cover piece,

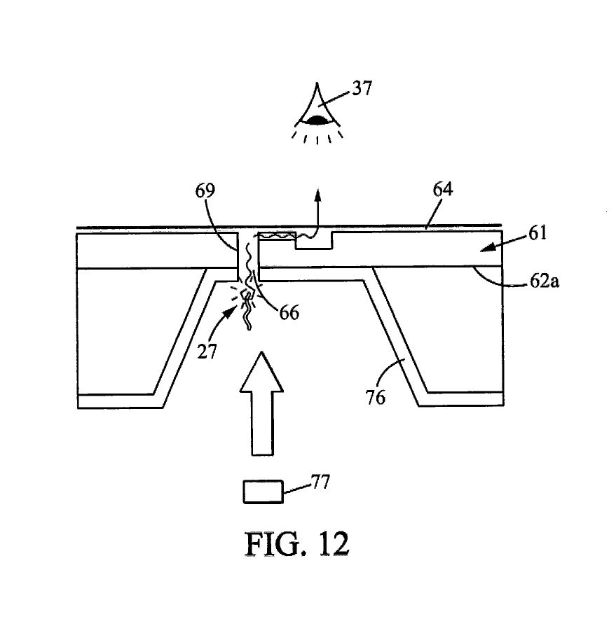

FIG. 12 is a sectional view of an embodiment of the cover piece, and

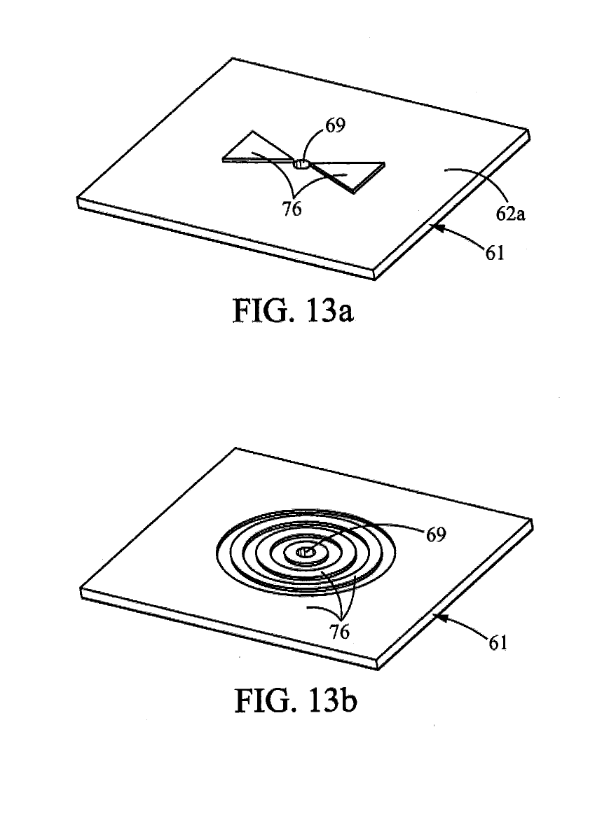

FIGS. 13a and 13b are perspective views of the underside of the cover piece.

In the various figures, the same references designate identical or similar elements.

FIG. 1 schematically represents an electrophoresis apparatus 1 according to a first embodiment of the invention. Such an apparatus has an instrument portion 2 and a computer device 3 connected thereto. The computer device 3 may act primarily to:

control the instruments of the instrument portion 2, and/or

receive data from the instrument portion 2.

The computer device 3 conventionally comprises a central processing unit 4 comprising a processor adapted to execute programs, random-access or read-only memory, etc. It also comprises user interfaces such as a keyboard 5a, a mouse 5b, and/or a screen 5c.

The instrument device 2 comprises a reservoir 6 containing a fluid adapted for performing electrophoresis in the reservoir 6. Such a fluid is for example a liquid solution.

The solution comprises, for example, anions and cations of the same salt, in high concentration, and particles in low concentration. The particles are objects at least hundred times greater in size than the ions of the solution, and well below one micron (for example less than 100 nm, or even 10 nm). Examples of particles are colloids, and macromolecules among which we can cite DNA molecules, RNA molecules, proteins, polysaccharides, and others. A low concentration of macromolecules is provided in the solution, these macromolecules being different from one another or identical, depending on the applications. The lowest concentration considered is one such macromolecule in the solution.

A membrane 7 in the reservoir 6 separates the reservoir 6 into two compartments 6a, 6b. The membrane 7 is provided in the reservoir 6 such that the exchanges of fluid between the first and the second compartments can occur only through passages 8 in the membrane 7. Depending on the application, one or more passages 8 are provided in the membrane 7. The membrane 7 is a synthetic, or artificial, membrane, meaning it is manufactured, as opposed to known porous biological membranes. In FIG. 1, a single passage 8 is schematically represented. The manufacture of the membrane 7 will be explained below.

FIGS. 3a (macroscopic) and 3h (microscopic) show an example of the synthetic membrane used for the first embodiment above. This membrane conventionally comprises a rigid base 22 which can be fixed in place in the appropriate position in the reservoir and contributes to the mechanical stiffness of the membrane. This base 22 is provided with a hole 23 which is for example a few microns in diameter in the XY plane transverse to the macroscopic direction of movement Z of the macromolecule between the two compartments 6a, 6b. The hole 23 is covered by a thin cover piece 24 fixed to the base 22 in any manner suitable for preventing the passage of species at the attachment. The cover piece 24 is for example slightly tensioned. The cover piece 24 is provided with at least one passage 8.

FIG. 3h shows an enlargement of the cover piece 24 at the passage 8.

The cover piece 24 comprises a body 61. The body 61 is for example made from a substantially rigid material of any suitable type, such as silicon, SiO2, SiC, or SiN. The body 61 has a thickness of less than a micron, for example about 0.1 micron. The body 61 may be translucent, in applications making use of optical detection.

The body 61 comprises a base section 62 having two opposite outer surfaces 62a, 62b. A pore 78 extends between the opposite outer surfaces 62a, 62b.

A channel bottom 63 extends in a main longitudinal direction X, being formed in the base section 62 at outer surface 62b. The channel bottom 63 has a width of about one third or less than the thickness of the cover piece 24, for example about one tenth or less than the thickness of the cover piece 24.

The channel bottom 63 opens into the pore 78.

The channel bottom 63 may open into a well bottom 73. The well bottom 73 has any suitable shape. It is formed in the base section 62 at outer surface 62b. The well bottom 73 has a width greater than the width of the channel bottom 63. This width may be at least two times the width of the channel bottom 63.

The cover piece 24 also has a covering sheet 64 assembled to the body 61.

The covering sheet 64 is assembled to the base section 62 while at least partially covering the outer surface 62b, and in particular at least partially covering the pore 78, the channel bottom 63, and the well bottom 73. The pore 78 is closed off on one side by the sheet 64. The channel bottom 63 and the covering sheet 64 thus together form a closed channel (or trench) 65. A depth p of the channel is approximately the width of the channel bottom 63 in a depthwise direction Z transverse to the main longitudinal X and widthwise Y directions. The channel 65 has a depth of about one third or less than the thickness of the cover piece 24, for example about one tenth or less than the thickness of the cover piece 24. This depth is for example less than 0.5 microns in the depthwise direction.

The well bottom 73 and the covering sheet 64 thus together form a covered well 74. The well 74 is in fluid communication with the channel 65 at an outlet 75. A depth p of the well is greater than the depth of the channel 65 in the depthwise direction Z transverse to the main longitudinal X and widthwise Y directions. The well 74 has a depth of about one half or less than the thickness of the cover piece 24, for example about one tenth or less than the thickness of the cover piece 24. The well 74 has a depth of about 1.2 times or more than the depth of the channel 65. This depth is for example less than 0.5 micrometers in the depthwise direction.

The covering sheet 64 comprises, in particular consists of, graphene, boron nitride (BN), or molybdenum disulfide (MoS2). The sheet 64 may be thin, in particular less than a nanometer thick, which enables easily creating a through-opening or pore 68 therein. The sheet is created for example as a two-dimensional crystal, of atomic-scale thickness.

The cover piece 24 has an inlet end 66 and an outlet end 67. The terms “inlet” and “outlet” are used in reference to the orientation of the cover piece 24 in the reservoir according to the present embodiment, but are illustrative only, as the cover piece 24 could alternatively be used for molecular movement in the opposite direction. The inlet and outlet ends 66, 67 are in fluid communication with the channel 65 and respectively with the first compartment and second compartment. In particular, the sheet 64 has a pore or through-opening 68 which opens into the well 74 and comprises the outlet end 67. The body 61 comprises the pore 78 opening into the channel 65 and having the inlet end 66. Pore 78 and pore 68 are offset from one another in the XY plane, and the inlet end 66 and outlet end 67 are offset from one another in the XY plane. Thus, the passage 8 comprises a first portion, substantially corresponding to pore 78 and extending in the Z direction, a second portion, substantially corresponding to the channel 65 and the well 74 and extending in the XY plane, and a third portion, substantially corresponding to pore 68 and extending in the Z direction.

The passage 8 has a transverse dimension D (in other words the transverse dimension of its narrowest portion) on the order of the dimension of the macromolecule being subjected to electrophoresis, but slightly greater than this transverse dimension of the molecule. Thus, different types of membrane can be produced having passages of different transverse dimensions D, according to the type of macromolecule to be analyzed. The dimension D is for example selected so that the transverse dimension of the macromolecule is between 0.5 and 0.9 times the transverse dimension D of the passage. The dimension D is for example about 25 nanometers or less, or possibly less than 10 nanometers or less. The dimension chosen depends on the size of the macromolecules to be studied. The thickness of the channel 65 is for example on the order of magnitude of the dimension D. Alternatively, it may be of an order of magnitude several times that of D.

A passage 8 of this size can be created using a focused ion beam technique for example.

FIGS. 3b to 3h illustrate an exemplary method for forming the cover piece 24.

As represented in FIG. 3b, one begins with a solid and intact body 61.

By focused ion beam (FIG. 3c), a through-hole 69 extending in the Z direction is created in the body 61, to form pore 78.

By focused ion beam (FIG. 3d), a channel bottom 63 is created in the body 61 in outer surface 62b, extending from the through-hole 69 in the X and/or Y directions. To do this it is sufficient to reduce the time of exposure of the body 61 to the beam, while imposing a relative movement between the body 61 and the beam to draw the desired path for the channel bottom 63. The length and geometry of the channel bottom 63 can be chosen on the basis of the application. The length of the channel bottom is at least on the order of the length of the macromolecule.

By focused ion beam (FIG. 3e), a well bottom 73 is created in the body 61 in outer surface 62b, opening into the channel bottom 63. To do this it is sufficient to increase the time of exposure of the body 61 to the beam, while imposing a relative movement between the body 61 and the beam to form the desired shape of the well bottom 73. The length and geometry of the well bottom 73 can be chosen on the basis of the application.

Then (FIG. 3f) the covering sheet 64 is joined to the body 61 at outer surface 62b, thereby closing off the channel 65 and the well 74.

Next (FIG. 3g), the sheet 64 is pierced at the well 74, thereby forming pore 68. This piercing can be done by focused ion beam, aiming to create as small of a hole as possible while remaining large enough to allow the passage of the macromolecule. The time of exposure of the cover piece 24 to the beam is reduced, to avoid piercing through the body 61 in this step. If the well 74 is of sufficiently large dimensions, it is unnecessary to have a high level of precision in the positioning of the piercing step. It is sufficient that the pore 68 is created so that it opens into the well 74.

One possible implementation of the microfluidic device just described is disclosed below. This is an implementation within the context of electrophoresis.

Arranged in the first compartment 6a is a first electrode 9a and arranged in the second compartment 6b is a second electrode 9b. The first and second electrodes 9a, 9b are part of an electrical system 10 adapted to generate an electric field in the reservoir 6 when said reservoir contains the solution. The electrical system 10 comprises an electric generator 11 connected by a pole to each of the electrodes 9a, 9b. The electric generator 11 allows applying a difference in potential between the electrodes 9a and 9b.

Also provided is a reading system 12. This is for example an ammeter connected in series between one of the poles of the generator 11 and the corresponding electrode. The reading system 12 is connected to the computer device 3, which records the intensity of the electric current flowing in the circuit.

The embodiment just described operates as follows. As represented in FIG. 1, the generator 11 applies a difference in potential between the electrodes 9a and 9b, which generates an electric field in the solution inside the reservoir. It may be arranged for example that at the beginning of the experiment, the macromolecules are all located in a given compartment, for example the first compartment 6a. The net charge of the macromolecule may be known in advance, which allows applying an electric field such that the macromolecule will be attracted by the second electrode 9b and will therefore pass through the passage 8.

As represented in FIG. 2a, for a given difference in potential V applied by the electric generator 11, the ammeter detects an electric current I as a function of time t. The electric current is for example about 10 nanoamperes (nA). Excluding the noise, the measured current is relatively constant except for a visible event 15 represented by a drop in the value of the current. Note the infinitesimal nature of this drop (about 0.4 nA), and its short duration (less than 0.1 seconds, usually a few milliseconds). Therefore ammeters 12 capable of detecting such low signal levels are used, as are generators capable of generating sufficiently constant voltages to enable detection of such a difference.

It is commonly accepted that this event corresponds to the migration of a macromolecule through the passage. One plausible explanation for this phenomenon is that the macromolecule, during its migration through the passage, essentially plugs it and therefore prevents the free flow of other ions of the solution that was taking place before the macromolecule entered the passage. This results in an increased electrical resistance of the solution, and therefore, for a given voltage level, a drop in the current I.

FIG. 2b highly schematically illustrates this phenomenon for a chromatin fiber. In the left window, a thin link 16 between two thicker clusters 17a, 17b passes through the passage 8 without substantially modifying the measured value of the electric current relative to the reference level (measured plateau 18 in the graph I(t)), while the passage of the cluster 17b causes a drop in the current (plateau 19 in said graph). The passage of a cluster 17c of intermediate size could correspond to an intermediate intensity between plateaus 18 and 19 (plateau 20). The graph of FIG. 2b is represented without any scale.

According to one embodiment, an electric field is applied and the migration of the macromolecule through the passage 8 is detected as explained above. As the length of the passage 8 is larger than when it is created essentially transversely to the cover piece, the period during which an electric current is detected as explained above in relation to FIGS. 2a and 2b is longer. Therefore, more information is obtained about the migration of the macromolecule through the passage 8.

According to one embodiment, a modulation system 13 is further provided for modulating the electric field. In this embodiment, the modulation system 13 is a general modulation system. It allows influencing the electric field throughout the reservoir. It comprises a feature where the electrodes 9a and 9b are reversible. An example of such electrodes is for example a pair of electrodes made of Ag/AgCl.

According to this embodiment, the modulation system also comprises an inverter 14 adapted to reverse the polarity of the electrical generator 11. The inverter 14 is also connected to the computer device 3 which can control the reversal.

When the central processing unit 4 detects that an event is occurring (it measures for example whether a period of time, during which the measured current is below a certain threshold relative to the reference current, exceeds a certain time limit), it can control the inverter 14 to reverse the polarity applied by the generator 11, as represented in FIG. 1a. Reversal of the potential applied by the generator 11 at time tᵢ will cause the reversal of the electric field inside the reservoir 6, such that the macromolecule 27 will now be attracted by electrode 9a. The modulation system is therefore activated by the reading system.

As can be seen in FIG. 2a, a second event 21 is then detected which may, as represented, be symmetrical to event 15 if the macromolecule has not had time to reverse.

By repeating the same process many times, we will therefore obtain numerous readings corresponding to the migrations of the macromolecule through the passage. These various readings can be added by the CPU 4 in order to increase the signal-to-noise ratio of the current measurement. An inverter is used which enables implementing such a reversal while presenting transient phenomena for a sufficiently short period of time at the scale of the macromolecule's passage migration time. This process is particularly advantageous if a single macromolecule is present in the solution.

In the above embodiment, one waits for the end of the migration of the macromolecule through the passage in order to perform the reversal. Alternatively, one could perform a systematic reversal before the end of the migration of the macromolecule through the passage, in one direction and in the other. By doing so, the experiment time is greatly reduced since the macromolecule is always present in the passage. However, very little information concerning the ends of the macromolecule might be obtained. By doing so, one could use a solution comprising a plurality of macromolecules, possibly different ones, which would be analyzed in turns.

With the invention, a passage 8 of great length is created, which allows increasing the time the macromolecule is present in the passage without increasing the thickness of the membrane. Moreover, as a major portion of the passage 8 is created at the surface, the macromolecule is easily accessible for detection (the sheet 64 is transparent to a certain number of radiations, in particular translucent, thus allowing optical detection of the macromolecule).

These advantages are also present when the electrophoresis method does not apply any reversals.

Alternatively, the channel 65 may be unvarying in shape (meaning it has a constant cross-section). Alternatively, as shown in FIGS. 3i and 3j, the width 1 of the channel may vary along the longitudinal direction thereof. Additionally or alternatively, the channel depth p may vary along the longitudinal direction thereof. These dimensional variations can be obtained by adjustments of the focused ion beam during the step of creating the channel bottom (FIG. 3d).

The channels represented above are longitudinal along direction X. However, any geometry in the XY plane can be considered.

A second embodiment of the invention will now be described in relation to FIG. 1 and FIG. 4. The apparatus substantially corresponds to the apparatus represented in FIG. 1, with the difference that the modulation system 13 of the second embodiment does not include the inverter 14 of FIG. 1, nor are the electrodes 9a and 9b reversible. According to the second embodiment, the system 13 for modulating the electric field in the reservoir is a local modulation system. It allows locally influencing the electric field present in the channel 65, at the passage. It comprises a local electrode 25 arranged on the membrane 7 (in particular on the cover piece 24) near the channel 65. The sheet 64 may then be made of an electrically insulating material, such as hexagonal boron nitride (h-BN). This allows the sheet 64 to electrically insulate the local electrode 25 with respect to other electrically conductive elements of the system. It could also be arranged that the sheet 64 is made by superimposing layers, therefore an electrically insulating layer (h-BN) is arranged opposite the electrode 25, and comprising an electrically conductive layer (graphene for example). In particular, a multi-layer of insulation (h-BN), conductor (graphene), insulation (h-BN) may be provided.

To clarify these concepts, with reference to FIG. 1 the membrane 7 comprises a surface 7b oriented towards electrode 9b located in the second compartment 6b, and a face 7a oriented towards electrode 9a located in the first compartment 6a. According to the embodiment of FIG. 4, electrode 25 is created on face 7b of the membrane 7. In this embodiment, the membrane 7, and in particular the body 61, is made of an electrically insulating material. The electrode 25 is for example created in the form of a layer of gold or platinum having a certain pattern. Specifically, the electrode 25 is created on the outer surface 62b of the body 61. The electrode 25 is interposed (in the thickness direction) between the body 62 and the sheet 64 (which is not represented in FIG. 4). This can be done for example by creating the electrode before the sheet placement step (3f), or even before the etching steps carried out by focused ion beam (in which case the pore 78, channel bottom, and well bottom can be created while also etching the conductive material of the electrode).

In another variant, the electrode 25 is created above the sheet 64 of electrically insulating material, as represented in FIG. 4a.

In another variant, the electrode 25 may be formed on the outer surface 62a opposite to that in which the channel bottom 63 is formed, as represented in FIG. 4b.

In the present exemplary embodiment, a set of two local electrodes 25, 26 is provided, arranged one on each side of the channel 63 and each connected to a local electric circuit 30. The two local electrodes 25 and 26 are placed in the local electric circuit at a different potential in order to form a capacitor. A local electric field is thus generated, and is superimposed on the general electric field applied by the generator 11. The electric circuit 30 also allows obtaining a reading of the capacitance. It is therefore connected to the computer system 3. The macromolecule 27 has a set of different segments from its first end to its second end, and in particular these vary the capacitance measured during their presence between the two electrodes 25 and 26. Thus, in addition to the reading system 12 which allows measuring the migration of the macromolecule through the passage, the system for modulating the electric field provides additional information during the migration of the macromolecule, due to the presence of the local electrodes 25 and 26.

Due to the length of the channel 65, multiple locations for the electrodes or electrode pairs can be provided along the channel.

Furthermore, when the electrodes 25 and 26 are sandwiched between the body 62 and the sheet 64, this sheet 64 electrically and chemically insulates the electrodes 25 and 26 from the solution.

The nanoscale electrodes 25 and 26 of the two embodiments are connected, where applicable, to the macroscale world (ultimately to the computer device 3) by microconnection systems.

A fourth embodiment is represented in FIG. 5. This embodiment includes elements of FIG. 4, in particular the cover piece 24 having local electrodes 25 and 26 on its surface 7b. These are connected to a local electric circuit 40 so as to generate a local electric field E_{y} extending in the Y direction at the channel 63, transverse to the migration of the macromolecule.

The application of a local electric field is used to influence the migration of the macromolecule through the channel 63 along direction X. The macromolecule 27 is composed of a succession of molecules each having a partial charge (negative, positive, or possibly zero) contributing to the net charge of the macromolecule which alone defines its translocation from one compartment of the reservoir to another. The local electric field E_{y} will induce an electrostatic force on these partial charges, which will be attracted and repelled by the edges 31, 32 of the channel 63. For example, a portion 33 of the macromolecule, which has a positive charge locally, will be attracted by the edge 31 of the channel 63. A mechanical interaction of friction of the macromolecule 33 on the edge 31 of the channel can thus occur, this friction contributing to slowing the macromolecule during its migration through the channel 63. As a result of this slowing, the event 15 at the detected signal of FIG. 2a will last longer and will therefore be easier to study. The application of the local electric field can be controlled by the computer system 3.

Depending on the local charge level of the macromolecule, and depending on the level of the electrostatic field applied by the local electrodes 25 and 26, it is possible not only to slow the macromolecule 27 during its migration through the channel 63, but even to immobilize it. The local modulation system thus makes it possible to define a molecular vise. Once the macromolecule is thus held in place, it is possible to subject it to any type of treatments and/or applications. The local system already allows measuring the force applied to the molecule in order to hold it in place. This force is additional data characteristic of the molecule at the blockade location. The measurement is sent to the computer system 3.

According to a fifth embodiment, represented in FIG. 6, one can combine the embodiments of FIGS. 4 and 5. Thus, at a first location of the channel, the capacitive reading system with electrodes 25 and 26 and the circuit 30 as embodied and described in FIG. 4 may be provided, and at a second location of the channel, the braking/blockade system with electrodes 35 and 36 and the circuit 40 as described in FIG. 5 may be provided.

According to a sixth embodiment, represented in FIG. 7, an electric field Eₓ is applied locally in the longitudinal direction X of the channel 65. This local electric field Eₓ is superimposed on the general electric field imposed by electrodes 9a and 9b. The local electric field thus applied is used to locally influence the electric field at the channel 65. One can thus produce a braking or blockade action on the macromolecule in the channel 65, not by mechanical friction as described above but by using the locally generated electric field to counteract the effects of the general electric field. To do this, a first electrode 45 is provided that extends to each side of the channel 65 at a first location, as well as a second electrode 46 that also extends to each side of the channel 65 at second location, and these two electrodes are connected by a generator 50 adapted to apply a difference in potential between them.

In the above embodiments, the electrodes may be created within the thickness, in accordance with the appropriate embodiments of FIG. 4, 4a, or 4b described above.

Where appropriate, the embodiments of FIGS. 4 to 7 are used in a membrane equipping the device 7 of FIG. 1. A general modulation system for modulating the electric field is then superimposed with a local modulation system.

According to a seventh embodiment, the electrophoresis apparatus described above is coupled with an optical detection system which detects the migration of the macromolecule through the passage 8. In particular, an optical detection system for detecting the presence and/or movement of the macromolecule in the channel 65 is used.

In this embodiment, as in the others, an optical sensor 37 is provided, visible in FIG. 1, adapted for optically detecting an image at the channel 65 on face 7b of the membrane. The optical sensor 37 is also connected to the computer device 3 in order to send the detected optical signals to it. If the sheet 64 is translucent, an event can be detected as long as it occurs in the channel 65, which is of substantial length.

According to a first variant embodiment, if the macromolecule is fluorescent, a confocal microscope can be used as the optical sensor 37 for imaging the channel, and detection enables counting the fluorescent molecules passing through the channel. The fluorescence may be generated by a light source 77 located on the side opposite to the optical sensor 37, and illuminating the inlet end 66. This light source 77 is for example a laser. An opaque layer may be provided in the body 61, for example assembled to the outer surface 62a without blocking the through-hole 69. The opaque layer may for example be a metal sheet 76 (for example of gold or gold alloy, for example TiAu less than a micron thick) assembled to the body 61 before the focused ion beam etching steps, and pierced during creation of the through-hole 69. An exemplary embodiment is shown in FIG. 12. The opaque sheet 76 blocks transmission of the excitation light in the direction of the optical sensor 37. The fluorescence is enhanced at the inlet end 66 by the presence of the sheet walls. Molecules 27 present in the channel 65 can be imaged by the optical sensor 37 through the translucent sheet 64, during a long period of time.

In yet another embodiment, FIG. 8 represents a portion of the outer surface 62a of the membrane around the pore 78. Patterns are provided for example in the form of metallization 76. The set of metallizations 76 formed around the pore 78 forms a certain optical pattern denoted with the general reference 34. For example, six metallizations are represented, arranged radially around the inlet end 66 and angularly equidistant from one another. However, any type of appropriate pattern is possible. FIGS. 13a and 13b show examples. In FIG. 13a, two sheets 76 of triangular shape are used, diametrically opposed with respect to the pore 78. In FIG. 13b, concentric rings around the pore 78 are used. These geometries enable defining plasmonic antennas at the inlet end 66 of the pore.

The optical pattern 34 can improve the optical excitation of the macromolecule by the light source 77 when it enters the passage 8.

Where appropriate, these optical detection systems can also be incorporated into the embodiments of FIGS. 4, 5, 6, and 7. Cooperation between the optical and electrical detections can provide better characterization of the molecule.

FIG. 9 shows a highly schematic depiction of a facility 51 for fabricating the body 61. For example, the through-hole 69 may be formed through a substrate 52 placed in a focused ion beam machining tool. The substrate 52 is intended to become the body 61. It is placed in a sample holder 53. A tip 54 is used to emit ions, for example such as gallium ions, which are extracted by an extractor 55 and focused by an electrostatic system 56 to drill the pore to the appropriate size in the substrate 52. Where appropriate, the substrate 52 is created beforehand by evaporating the conductive metal on one or more surfaces of the insulating substrate and then defining the traces by lithography.

The channel bottom 63 and the well bottom 73 can be created by the same type of operation on a surface of the substrate, by a relative displacement of the beam and substrate and adjusting the exposure time.

Fabrication by focused ion beam makes it possible in particular to obtain stable geometries compatible with the desired application while reducing the risk of blocking the passage and while providing a relatively steep passage edge. Synthetic passages are more easily integrated. In addition, the ions used, such as gallium ions, can pierce the insulating substrate as well as the metal layer located in the surface above and/or below, depending on the embodiments. The process is highly reproducible (variations of about 2-5%).

In the above embodiments, the membrane 7 physically separates the two compartments 6a, 6b. Alternatives are possible, however. One exemplary alternative is provided in FIG. 10. In this alternative, the compartments 6a, 6b are arranged on the same side of the cover piece 24. The compartments 6a, 6b are separated by a wall 72. The inlet 66 and outlet 67 ends are formed one on each side of the wall 72. A cover piece 24 can be obtained by employing the manufacturing method described above until the step of FIG. 3e, where the well bottom 73 is now pierced through. The sheet 64 is assembled as shown in FIG. 3f, but it is not pierced. Then the wall 72 is mounted on face 62a of the cover piece.

In another alternative, as shown in FIG. 11, the device described above could incorporate its own compartments 6a, 6b. The compartments 6a, 6b could be directly created as recesses in the body 61, one on either side of the channel 65. This could be done according to the above method, but stopping at the step of FIG. 3f and replacing the step of generating the through-hole 69 by fabricating a blind hole (which is possible if the body 61 is sufficiently thick and/or by adjusting the process of etching the holes 69, 74). The blind holes can then serve as a reservoir.

Thus, in this example, the channel opens into an inlet and/or outlet reservoir formed in the body.

The covering sheet covers the inlet and/or outlet reservoir so as to close it/them.

In this case, the system is substantially sealed, and it is possible to miniaturize the electrical system 10 for embedding in the device.

In the above examples, movement of the molecules is generated by electrical action. However, additionally or alternatively, other technologies are possible, such as controlling the hydrostatic flow (suction for example), gravity, etc.

Envisaged applications include the analysis of proteins for diagnosis, drug development, identification of molecules for security and defense applications and environmental protection, desalination of sea water, generation of electric or hydraulic energy.

It is possible to create several similar passages in parallel within the same membrane 7, each according to an example described above.

1. A microfluidic device, wherein the microfluidic device comprising a body and a covering sheet,
the body comprising a base section having an outer surface, a channel bottom extending in a main longitudinal direction being formed in the base section in the outer surface, the channel bottom being formed by focused ion beam,
the covering sheet being joined to the base section while at least partially covering the channel bottom, thereby forming a channel.

2. The microfluidic device according to claim 1, wherein the covering sheet comprises an electrically conductive layer and/or an electrically insulating layer, for example a superimposed electrically conductive layer and electrically insulating layer, in particular wherein a layer of the sheet comprises, in particular consists of, graphene, boron nitride, or molybdenum disulfide.

3. The microfluidic device according to claim 1, wherein the body comprises, in particular consists of, silicon or an oxide, carbide, or nitride of silicon.

4. The microfluidic device according to claim 1, further comprising at least one electrode at least partially arranged in the vicinity of the channel.

5. The microfluidic device according to claim 1, comprising an inlet end and an outlet end, both in fluid communication with the channel.

6. The microfluidic device according to claim 5, wherein the inlet end and/or outlet end is part of a pore traversing the body and opening into the channel and extending in the thickness direction.

7. The microfluidic device according to claim 1, wherein the channel opens into an inlet compartment and/or outlet compartment formed in the body.

8. The microfluidic device according to claim 7, wherein the covering sheet covers the inlet compartment and/or outlet compartment.

9. The microfluidic according to claim 1, wherein the depth and/or width of the channel varies along the main longitudinal direction.

10. The microfluidic device according to claim 1, wherein the body is a thin body less than 10 microns in thickness.

11. The microfluidic device according to claim 1, wherein a well bottom is formed in the base section at the outer surface the well bottom being formed by focused ion beam,
the covering sheet being joined to the base section while at least partially covering the well bottom, thereby forming a well in fluid communication with the channel.

12. An apparatus, wherein the apparatus comprises:
a reservoir adapted to receive an electrically conductive solution,
a microfluidic device according to claim 1, immersed in the reservoir and separating the reservoir into first and second compartments, the channel being in fluid communication with the first and second compartments to permit fluid communication between the first and second compartments,
a transport system adapted to generate movement of the solution in the reservoir when the latter contains the solution,
a system for characterizing the species contained in the reservoir.

13. The apparatus according to claim 12, wherein:
the transport system comprises an electrical system adapted to apply an electric field in the reservoir when the reservoir contains the solution,
the characterization system comprises a system for reading the electric field in the reservoir.

14. The apparatus according to claim 12, further comprising a modulation system for modulating an electric field present in the channel.

15. The apparatus according to claim 13, wherein the electrical system comprises first and second electrodes respectively arranged in the first and second compartments, between which the electric field is applied,
wherein the modulation system comprises said first and second electrodes which are reversible, and an inversion system adapted for reversing the polarity of an electric field applied between the two electrodes.

16. The apparatus according to claim 14, wherein the microfluidic device further comprises at least one electrode at least partially arranged in the vicinity of the channel, wherein the modulation system comprises a set of local electrodes comprising at least said electrode, and a generator adapted to apply a local electric field at the channel via said set of at least one local electrode.

17. The apparatus according to claim 12, further comprising an optical reading system adapted to take an image of the channel.

18. The apparatus according to claims 11, comprising at least one of the following characteristics:
the microfluidic device comprises a single passage,
the solution has a high concentration of solute and a low concentration of particles, the particles being potentially identical or possibly even a single particle, the transverse dimension of the particles possibly being between 0.5 and 0.9 times the transverse dimension of the chan
